(19) United States
(12) Patent Application Publication
(10) Pub. No.: US 2005/0107771 A1
(43) Pub. Date: May 19, 2005

(54) Device for draining urine from the human bladder

(76) Inventor: Rudolf Finkbeiner, Niederstetten (DE)

Correspondence Address: M. Robert Kestenbaum, 11011 Bermuda Dunes NE, Albuquerque, NM, 87111, US

(21) Appl. No.: 10/988,916

(22) Filed: Nov. 15, 2004

(30) Foreign Application Priority Data
Nov. 15, 2003 (DE) 103 53 618.3

(51) Int. Cl. A61M 5/00; A61M 25/00; A61M 27/00

(52) U.S. Cl. 604/544

(57) ABSTRACT

A device for voluntary, controlled urethral drainage of urine from the bladder of a human suffering from urinary incontinence has a tube arrangement that conveys human body fluid and is made of plastic and is physiologically compatible, and has an actuating device for this tube arrangement. The tube arrangement is a urinary catheter tube whose length corresponds to the length of the path between the bladder outlet and the urethral outlet and whose external dimension corresponds to the width of the urethra. The catheter tube protrudes beyond this length with an excess length.

### Cross References to Related Applications

Not applicable.

### Statement Regarding Federally-sponsored Research/Development

Not applicable.

### BACKGROUND OF THE INVENTION

1. Field of the Invention

The invention relates to a device for voluntary, controlled urethral drainage of urine from the bladder of a human suffering from urinary incontinence, with a tube arrangement which conveys human body fluid and is made of plastic and is physiologically compatible, and with an actuating device for this tube arrangement.

2. Technical Field

A device of this kind has for many years been produced and sold by the company AMS. With this kind of device, the sphincter of the bladder is replaced by a cuff which surrounds the urethra. The cuff is filled with a fluid and surrounds the urethra with gentle pressure so that, in this state, the urethra is surrounded at a point situated a distance downstream from the location of the sphincter. To empty the bladder, pressure is applied to a switch device which contains valves and resistance parts. The cuff is connected via a tube to the control pump. A further connection on the control pump leads via a further tube, which is reinforced against compression, into a pressure-regulating balloon. The latter regulates the pressure exerted on the urethra by the closure cuff.

Once the urine has been eliminated, the fluid then flows from the pressure-regulating balloon back into the cuff. The pressure in the cuff rises again and in this way the urethra is once more closed.

A disadvantage of this system is that all the components, namely pressure-regulating balloon, cuff, kink-resistant tubes, and control pump, have to be implanted. The patient squeezes the pump several times to make the fluid flow from the cuff into the balloon.

After some time, the fluid flows back into the cuff from the balloon. The control pump is implanted in the scrotum so that the pump can be squeezed and the above-described automatic procedure is triggered.

The disadvantage of this is that the whole device has to be implanted in the patient's body and the actuation of the control pump requires a certain amount of practice. The system is filled with, and remains filled with, a saline solution. Even though the pressure exerted on the urethra by the cuff is gentle, this can nevertheless lead to complications, for example swelling.

If parts of this device need to be replaced, a surgical intervention is required. Urine not only remains lying in the bladder, but also in the urethra as far as the cuff, and a thin film of urine also remains in that area of the cuff which shuts off the urethra. Nor does the known device in any way prevent proliferation of tissue on the natural sphincter muscle.

Finally, the known device can be activated for the first time only after the surgical implantation wound has completely healed.

By far the most common way of dealing with the problems of incontinence is to use highly absorbent underwear inserts like sanitary towels, or also diaper pants which themselves constitute an absorbent insert. These diaper pants may in some cases have elastic garters which surround the wearer's thighs, and also the hip region.

Finally, there are diaper pants and inserts or briefs which function with granular material that has been developed for large amounts of urine. However, these devices are expensive. In order to make savings, many patients try to increase the urine absorption volume even further by additionally using normal inserts, or additionally using highly absorbent paper.

The devices used here must be adapted to the bladder size of men on the one hand and of women on the other. Moreover, account must be taken of the different anatomical conditions in men on the one hand and in women on the other, so that different devices have to be used for both sexes. Finally, account must also be taken of the bladder weakness group in which the patients are to be classified, and measures have to be taken accordingly. For example, a diary should be kept recording urination routine. This diary has several entry columns. The day of the week and date are to be entered under “Day”. The times at which drinks are taken and the amount of liquid in ml are also to be entered, and a column is provided to record the urge to urinate. There is another column for recording urination in ml, then loss of urine classified by amount (small, moderate, severe), and also change of sanitary towel.

The devices have to be adapted to the different forms of incontinence, such as overflow incontinence, stress urinary incontinence, urge incontinence, reflex incontinence, etc.

Over and above these types of incontinence, there is also the question of whether the patient has complete or partial control over the voluntary or involuntary nature of evacuation of the bowels.

In men at least, there is often a secondary effect caused by the fact that the urine often penetrates into the space between glans and foreskin, which causes more than painful swelling and constitutes a serious health problem. This is because the urine very rapidly decomposes.

Finally, the absorbent articles have to be manufactured, transported and also disposed of. For the patient, the problem of manufacturing and transport is mostly “only” a money problem. However, in these respects too, the patients also have to make considerable contributions.

The disposal of the used sanitary towels, briefs, etc. is, however, extremely difficult because, at least in Germany, these have to be disposed of as separate waste. Over and above all of this are the prostate problems in men, and the fact that in women the urethra is very short, so that particular care has to be taken. In particular also because bladder weakness occurs to a greater extent, for example due to childbirth.

That the AMS device could be used in women is unknown to the inventor.

The object of the invention is to avoid at least some of the abovementioned disadvantages, so that men and women are able to live a more tolerable and dignified existence.

For example, many of the previously used absorbent devices have an outer casing of plastic film. When the patients move, this film makes a noticeable rustling noise, so that many patients have got into the habit of carrying around paper handkerchiefs and their packaging, so that the patients, for the purpose of disguising the noise, can now and then rustle the packaging of the paper handkerchiefs.

The inventor could easily mention other taboo areas which have to be taken into consideration by patients who are thus afflicted.

The invention is also intended to permit X-ray examination of body parts, to make it easier for the patient to cope with his/her condition, to facilitate the work of doctors and nursing staff, and, if appropriate, to provide improvement without the patient having to perform disciplined physical exercises.

### SUMMARY OF THE INVENTION

According to the invention, the above object is achieved as follows:

The tube arrangement is a urinary catheter tube whose length corresponds to the length of the path between the bladder outlet and the urethral outlet and whose external dimension corresponds to the width of the urethra, and which catheter tube protrudes beyond this length with an excess length.

In the area of its excess length, the catheter tube has a urine-tight shut-off element, which can be opened and closed and has a long period of durability.

The invention thus starts out from a catheterization tube which at one end reaches into the bladder, lies in the urethra and protrudes via the urethral opening. In so far as the catheter tube protrudes beyond the end of the urethra, a shut-off element is provided there which the patient or a helper can open or close. When the shut-off element is closed, no urine flows from the catheter tube. When the shut-off element is opened, urine flows from the urethra practically in the same way as in healthy individuals. Thus, the sphincter muscle has, as it were, been displaced into the area of the excess length of the catheter tube. The excess length of the catheter tube can be of a small dimension. In males, it is sufficient for the excess length to cover a short distance so that the patient or a helper can open and/or close the shut-off element. A few centimetres are sufficient for this, so that the patient is able to wear normal clothing. This also applies to leisurewear, including snug swimming trunks. In the case of females, it suffices if the excess length extends slightly past the vulva.

The catheter tube and the catheter tube excess length are made in one piece. These features of the invention ensure that both the catheter tube and the excess length of the catheter tube can be introduced into and removed from the urethra as a single tube. The one-piece configuration can also be obtained by welding, adhesive bonding or another type of connection if one wishes to give the catheter tube excess length other properties, for example arrangement of the shut-off element, sufficient softness of the catheter tube excess length material for clamping and shut-off elements, etc.

The catheter tube and the catheter tube excess length have the same external contour. These features of the invention ensure that no inconvenient steps or shoulders are present between the two catheter tube parts.

They have the same internal contour. These features of the invention ensure that the flow behaviour is the same both in the catheter tube and in the catheter tube excess length.

The catheter tube, in its end area remote from the shut-off element, has a position-securing device. These features of the invention prevent the catheter tube from moving out of the ureter. This also ensures that the end area with its opening device is permanently situated inside the bladder.

The position-securing device is a circumference-enlarging element. These features of the invention permit use of the well-established, gentle and safe balloon element. As is known, this balloon element can be inflated when the end area of the catheter tube is situated inside the bladder at the desired site. In addition, when withdrawing the catheter tube, the balloon element can first be emptied of air so that it bears on the circumference of the catheter tube. For this purpose, the catheter tube is provided with air channel devices which in a known manner lead as far as the balloon element. At the outer end area of the catheter tube, an air valve is provided, to which a conventional syringe can be attached for filling the balloon with air and for emptying the balloon.

The circumference-enlarging element is an inflatable balloon element. These features of the invention mean that a simple shut-off element can be used. The actuation of stopper elements can be easily learnt, is known to very many people from other areas of use, can be produced inexpensively and can readily be made liquid-tight.

The stopper element has a grip edge. These features of the invention ensure that the stopper can be easily handled via the grip edge. It does not matter if a stopper is lost by mistake, for example in a toilet bowl. Other stopper elements can easily be kept ready, since they can easily be produced in a large number.

The grip edge has a limit stop. These features of the invention ensure that the stopper element cannot be pushed too far into the catheter tube excess length and consequently the tube is not overstretched in this area. In addition, the catheter tube can also be kept sterile with the stopper element.

The grip edge also has a sealing action relative to the free end of the excess length. These features of the invention mean that the inside of the grip edge can also be used for sealing purposes, so that a radial, circular sealing surface is created which can provide perfectly good sealing, because a sealing action is needed only in respect of liquid, not in respect of gaseous media.

The stopper element has circumferential ribs on its stopper. These features of the invention ensure an even better sealing action, because the circumferential ribs have a still better sealing action which is better than flat sealing surfaces, for example of a cylinder with respect to a cylindrical inner wall of the catheter tube. Static over-determination with multiple sealing surfaces can be avoided with elastic material.

The shut-off element is a spring element having the form of spring clips. These features of the invention mean that the stopper can be screwed into the free end of the catheter tube or the catheter tube excess length. Performing this screwing procedure does not demand too much of the patients, which is especially useful when things are “urgent” in a toilet with often poor lighting and confined space.

Moreover, the invention allows patients to bathe, swim and carry out other sporting activities practically as normal.

The shut-off element is a spring element having the form of spring clips. These features of the invention mean it is necessary simply to provide a spring element, which can have a design which the patient has also learnt how to deal with in the past, for example with tie holders, napkin holders, office articles, clothes pegs with leaf springs, etc.

The spring clip is in the nature of an arterial clip. By means of these features of the invention, a simple spring clip is obtained of a type which has long been known, and also in a suitable size and with the other necessary properties. Such arterial clips are easy to operate, do not rust, do not hang down, are inconspicuous, and, for people working in the health sector, actuating such arterial clips is a routine manoeuvre.

The spring clip has the form of a paper clip. By means of these features of the invention, a spring element is obtained which is sufficiently reliable, costs little, is easy to produce, and whose use is routine for most people.

The spring clip has the form of a small fold-back clip. These features of the invention afford the same advantages, but additionally a broader clamping surface. The user knows or very quickly learns how to open the clamping jaw via the actuating arms of this type of clip.

The shut-off element has the form of a nozzle with rotatable plug. These features of the invention likewise afford a simple shut-off element whose mode of action is also readily apparent without long explanations and an accompanying handbook.

The shut-off element has the form of a closure cuff of the AMS type. By means of these features of the invention, it is possible also to apply the previous experience gained from otherwise implanted devices.

The “artificial bladder sphincter” of the type AMS 800 is not implanted. These features of the invention mean that the previously used device can also be used here with very minor modifications, but without implanting it. Many patients are familiar with this device and in practice do not have to be taught how to use it.

The shut-off element is a screw with screw head, which screw can be screwed an internal thread in the end area of the excess length. These features of the invention provide for a shut-off element which can be screwed into the end area and thus has the sealing action of a stopper screw. However, care must be taken to ensure that a sufficiently great counter-torque can be applied to the excess length in order to compensate for the screwing-in torque.

The shut-off element is a valve. These features of the invention open up the use of valves which only have to be liquid-tight and do not have to be airtight. There are a number of readily usable shut-off elements with which people are already familiar.

The shut-off element is a valve which is of the kind used in a water wing, in inflatable toy animals, or in beach balls, or in similar devices, and opens under a radial actuation force. These features of the invention disclose such a shut-off element of a type which is mass-produced. Here, it is simply necessary that the patient can easily open the shut-off element. In this kind of valve used in water wings, this is done by squeezing the valve together at its circumference, as is also done when removing air from inflatable toy animals, beach balls and water wings or the like.

A securing stopper is provided at the end of the valve. A double safeguard is obtained by these features of the invention these securing stoppers are for the most part moulded integrally on the valve tube and cannot therefore be lost. In addition, the pressures in the ureter are much lower than in water wings, for example, and, in addition, the seal only needs to be liquid-tight, it does not have to be airtight. Such valves are produced by numerous companies, for example by Holzapfel from 37253 Eschwege, Germany. Such valves are called lip valves and are used in life jackets, inflatable chairs etc. Whereas respiratory air pressures have to be maintained in these, the liquid pressures in the bladder and urethra are very much lower.

In the pinch area of the catheter tube excess length, the tube material can be pinched until fluid is closed off, but is elastically resilient. These features of the invention ensure that only a slight force is needed to pinch off the catheter tube excess length, but the material of the catheter tube excess length, at least in the area of the closure element, returns to its previous cross section dimension.

### BRIEF DESCRIPTION OF THE DRAWINGS

The invention is now described with reference to preferred illustrative embodiments. In the drawings:

FIG. 1 shows a diagrammatic side view of the prior art,

FIG. 2 shows a side view, analogous to FIG. 1, for a first embodiment of the invention,

FIG. 3 shows an enlarged view of the invention, compared to FIG. 2, with more precise depiction of a catheter tube excess length,

FIG. 4 shows a view similar to FIG. 3, but with another variant in this area,

FIG. 5 shows another variant in the area of the catheter tube excess length, partially in cross section,

FIG. 6 shows a view, like FIG. 5, but illustrating another variant of the invention,

FIG. 7 shows the side view of a small clothes peg, which corresponds to pinching of an area in the area of the catheter tube excess length,

FIG. 8 shows a view of a small fold-back clip,

FIG. 9 shows a diagram of a lip valve, as is used for children's water wings, balls, inflatable toy animals, etc.

### DETAILED DESCRIPTION OF THE INVENTION

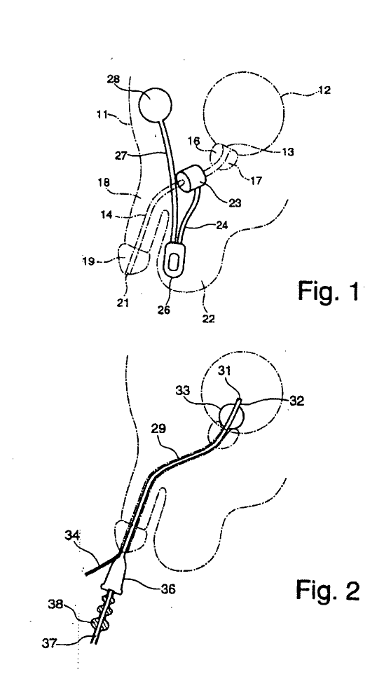

FIG. 1 is a diagrammatic view of the prior art:

Fully implanted in a body, whose contour 11 is indicated here, there is a bladder 12, shown by broken lines. The two associated ureters coming from the kidneys are not shown.

At the outlet 13 of the bladder 12, the latter merges into a urethra 14, which is shown by broken lines, likewise downstream of the outlet 13 the sphincter muscle 16 and the prostate 17. The sphincter muscle 16, in the contracted state, closes the bladder in so far as said sphincter muscle shuts off the urethra there. In a healthy individual, the prostate therefore is not permanently connected to the bladder. Only when the sphincter muscle opens, voluntarily, is there a flow through the prostate, and it is also designed so that it is not permanently subject to the internal pressure of the bladder 12.

The urethra 14 follows.

The urethra 14 extends through the penis 18, onwards through the glans 19 as far as the tip 21 of the glans.

The foreskin is not shown here. It surrounds the glans in a known manner.

The contour 11 merges in a known manner into the scrotum 22. The anatomical conditions are in this respect known.

In females, the anatomical conditions are also known. As regards incontinence, the female anatomy differs from the male anatomy to the extent that the urethra is very much shorter and ends directly in the vagina.

In the known device, a closure cuff 23 is provided which is connected via a tube 24 to a control pump 26. From the latter, a second tube 27 leads to a pressure-regulating balloon 28. The balloon 28, the tubes 24, 27, the closure cuff 23 and the control pump 26 are filled with physiological saline solution. When the patient wants to empty the bladder 12, he presses, if appropriate several times, on the control pump 26 at the suitable location of the scrotum 22. Thereupon, the physiological saline solution flows from the closure cuff 23 via the first tube 24 through the control pump 26 and via the second tube 27 into the pressure-regulating balloon 28. The closure cuff 23 thus permits through-flow, and the bladder 12 can be emptied.

After a certain time, the flow takes place in the reverse direction, the closure cuff 23 is pressurized, and the urethra is once again closed.

In so far as the device in FIG. 1 is shown with continuous lines, it can also be used in the invention outside the body, without implantation, in patients who have become accustomed to this type of bladder control.

FIG. 2 shows the same anatomical conditions, but without implanted parts. Here, a urine catheter tube 29 is guided from the tip 21 of the glans into the urethra 14 and passes through the prostate and the sphincter muscle. The upper end area 31 has an opening 32 for urine drainage.

The scale in FIG. 2 is not accurate, since the opening 32 lies nearer to the sphincter muscle 16, so that the residual urine in the bladder 12 in the usual way remains low.

However, after correct positioning of the opening 32, and in order to ensure that the latter cannot inadvertently be drawn downwards, a very thin-walled balloon torus 33 is provided at a distance from the end area 31, and this balloon torus 33 can be inflated to a considerably greater diameter than the catheter tube 29. The inflation is effected via a thin air channel (not shown) which, in the catheter tube 29, extends from an air channel attachment 34 to approximately the middle of the balloon torus 33. Upon insertion of the catheter tube 29, the balloon torus 33 lies in the manner of a very thin skin, and barely perceptible, on the circumference of the catheter tube 29 just behind the end area 31. When the opening 32 has reached its correct position inside the bladder 12, a pump device is attached to a valve end-piece (not shown) and the balloon torus 33 is pumped up from there. It does not in any way conceal the end area 31 and, above all, the opening 32, but prevents a situation in which the catheter tube 29 can be pulled downwards and, in so doing, the opening 32 can become covered, with the result that the tube interior of the catheterization tube 29 could no longer be used for draining the urine.

When the catheter tube 29 is pulled downwards and out, for example in order to insert a new catheter tube after a few months or weeks, a suction pump is attached to the valve outlet (not shown) of the air channel attachment 34. By means of the under pressure, the balloon torus 33 again bears on the outer surface of the catheterization tube 29 and offers no resistance to withdrawal.

The catheterization tube 29 has a connector piece 36 at its lower end, located downstream of the air channel attachment 24 and also outside the tip 21 of the glans. This connector piece has the form of a spout which, at the top, communicates with the inside of the catheter tube 29. A further catheter tube 37, which is short however, is now joined to the connector piece 36. The catheter tube 29 thus continues in the form of a catheter tube excess length 37. A liquid-tight connection is provided by a suitably configured mating piece 38 which, in its top area, communicates with the inside of the catheter tube 29 and thus also with the opening 32.

The liquid-tight connection between the connector piece 36 and the mating piece 38 can be a plug connection on the basis of elastic material, so that this connection is maintained by force fit and/or form fit. However, if one opts for a one-piece continuation of the catheter tube 29 in the form of a catheter tube excess length 37, then it is not necessary to have any complex connector piece 36 or mating piece 38. Instead, in this case, a simple, liquid-tight connection suffices, for example obtained by adhesive bonding or plastic welding or laser welding or the like.

In its simplest form, one illustrative embodiment of the invention entails using a sealing stopper 39 which has a head 41 which a patient can grip easily and secure against slipping. The shaft 42 of the sealing stopper 39 has the form of a pointed cone corresponding to the opening angle of the connector piece 43. Located on the shaft 42, and formed integrally with it, there are sealing ribs 44, 46 which bear with a liquid-tight fit on the inside wall of the connector piece 43. However, the sealing stopper 39 is short enough to ensure that a point does not protrude too far into the catheter tube excess length, which would mean that the sealing ribs 44, 46 would bear with too little pressure, or with no pressure at all, on the inner wall of the connector piece 36. Moreover, the head 39, with its annular rear face 47, maintains a distance, or at least a sufficient distance, from the lower edge 48 of the connector piece 43 to ensure that, although the head 39 can be gripped easily, there is no static over-determination which would reduce the contact pressure of the sealing ribs 44, 46 on the inside wall of the connector piece 43.

Such sealing stoppers 39 are easy to produce. If the patient loses them, their replacement costs very little or nothing.

Between the upstream end area 56 and a limiting rib 57 located just after this, the catheter tube excess length 49 is elastic, but designed to recover its shape. This is the tube piece 58. This tube piece 58 is shown in the unstressed state. In a further illustrative embodiment, as shown in FIG. 4, it is closed, for example, by paper clip U-shaped springs, by arterial clips, whose configuration and effect are generally known, by pegs like the one in FIG. 6, by fold-back clips which are known per se and opened using their actuating arms, or similar devices.

According to FIG. 4, in this illustrative embodiment the catheter tube 29 continues in practically the same form in the catheter tube excess length 49. However, the catheter tube excess length 49 can here be connected in the same way as the catheter tube excess length 37, i.e. they can be connected, in the manner seen from FIG. 2, to a connector piece 36 or a connector piece 43. According to FIG. 4, the catheter tube excess length 44 has, after the glans tip 21, two annular ribs 51, 52 which are arranged at a distance from one another. A spring element sits on the elastic tube area 53 between the annular ribs 51, 52.

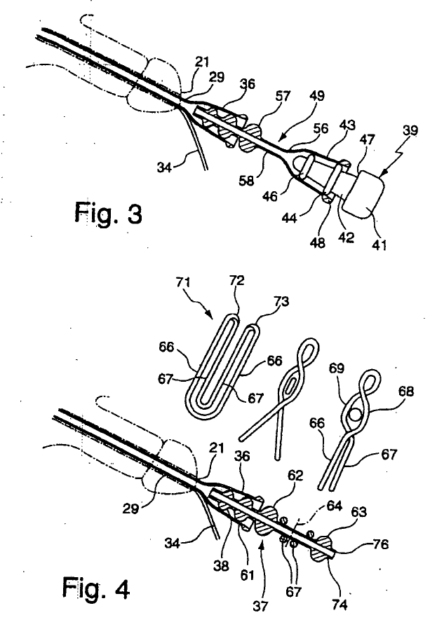

A further illustrative embodiment of the invention, according to FIG. 4, again has the connector piece 36, a mating piece 61, a limiting rib 62, analogous to the limiting rib 57 from FIG. 3. Instead of the end area 56 which also has a limiting purpose in the preceding illustrative embodiment, a second limiting rib 63 is provided here. At least between the limiting ribs 61, 63, a tube piece 64 is provided here, which is analogous to the tube piece 58 and which can be closed for a longer time. This is shown diagrammatically in the middle figure of the spring element. To cancel the liquid barrier, the branches spread apart in the closed state are pressed together, as the right-hand figure shows, in which the branches 66, 67 are almost parallel to one another and the bulges 68, 69 thus no longer exert any pressure on the tube piece 64. This spring element 71 applies the spring force essentially from the U-bends 72, 73. The actuation here essentially involves the branches 66, 67 being released or, according to the right-hand figure, being squeezed together between for example thumb and index finger.

As will be seen, practically the entire length of the tube piece 64 is used here and is shut off in the closed state immediately after the limiting rib 62.

Since the spring element 71 has a round profile at least towards the tube piece 64, the tube piece 64 is not in any way punched.

The two limiting ribs 62, 63 provide security against axial movement, so that the spring element 71 with its bulges remains on the tube piece 64 both in the shut-off state and also in the open state.

Since the spring element as a whole is wide, that is to say the distance between the branches 66 is large and both pairs of branches 66, 67 are perpendicular to the tube piece 64, the actuation is much more free of torque than in the case of likewise like-wise usable spring elements which have the form of simple U-shape springs.

Since the tube piece 64 is soft, it cannot completely filter out inexpertly applied forces and torques, but it can considerably reduce these.

Where the catheter tube excess length 37 extends inside the limiting ribs 62, 63, the tube walls are not influenced, so that the tube piece 34 in the area of the limiting ribs 62, 63 has essentially the same shape, namely permits flow of liquid. The limiting ribs 62, 63 are made in one piece with the catheter tube excess length 37 and form a part thereof.

Downstream from the limiting rib 63, the catheter tube excess length 37 continues with a very short tube section 74 which is cut radially at its free end face, is open and has a detachment edge 76 which permits a smooth flow of urine, which does not break up, and thus a directionally precise release of urine.

Instead of the spring element 71, it is also possible to use the closure cuff 23 from FIG. 1. This too is held more or less axially immovably by the limiting ribs 62, 63. The tube 24, the control points 26, the second tube 27 and the pressure-regulating balloon 28 can now be provided outside the body, that is to say outside the contour 11. No surgery is thus needed. The control points 26 can be provided at another fixed position and outside the scrotum 22, which also applies to the second tube 27 and the pressure-regulating balloon 28. If appropriate a tube 24 is chosen which is much longer, as shown in FIG. 1 or as is customary, so that the diaphragm pump can be provided, for example, in a pocket or in a suitable undergarment part. Since the closure cuff 23 known per se is constructed in the same way as the cuff of a blood pressure meter, but has a miniaturized format, the closure cuff 23 can be placed in the correct position onto the tube piece 64 which has the softness of the natural urethra 24.

One could furthermore provide the closure cuff 23 on the tube piece 64, so that here too the tube piece 58 can be opened and closed in the manner just described.

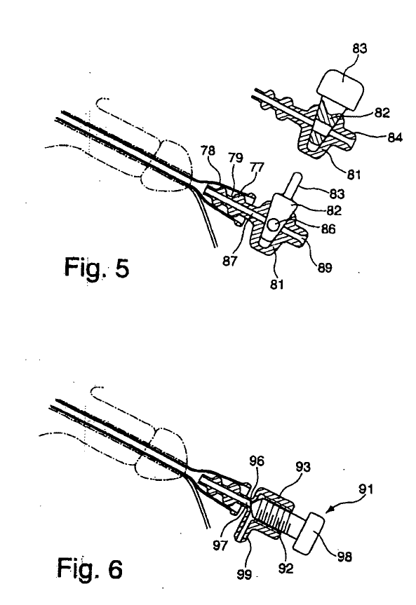

According to a further illustrative embodiment, sealing ribs 77, 78 are provided on a connection piece 79. This connection piece merges to the right into a nozzle 81. In whose conical interior a plug sits. It can be turned via a rotatable grip 83, but cannot be pulled upwards. To the right, the nozzle 81 merges into an outlet 84. In the position of the plug 84 shown in the bottom part of FIG. 5, the plug provides a seal to the outside because its transverse bore 86 is not aligned with the conduit sections 87, 89. However, in the position shown in the upper part of the figure, liquid can flow from the conduit section 87 through the transverse bore 86 and into the conduit section 89, and the bladder 12 can thus empty.

In the illustrative embodiment according to FIG. 5, there is no deflection of the flow of urine inside the nozzle 81.

In the illustrative embodiment according to FIG. 6, some of the details do not have to be explained again. Here, a threaded screw 91 is screwed with its threaded shank 92 into a jacket 93 which has an internal thread 94. In the position shown, the shank point 96 closes the conduit section 97. The shank point is in this case a conical point. If the screw head 98 is turned in the opening direction, the right-hand end of the conduit section 97 is freed and the urine can flow downwards out of the connection piece 99. A very precise regulation of the flow of urine is possible here.

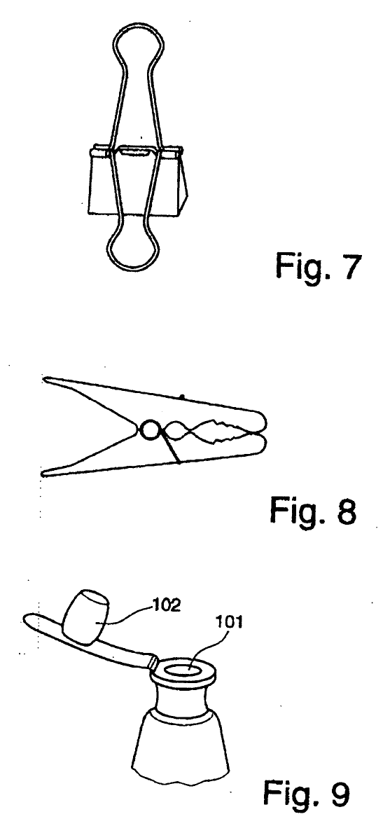

FIGS. 7 and 8 show very small, inexpensive closure means whose operation is familiar to many people. FIG. 7 is a perspective view of a spring clip. If its two steel arms are folded back, as is shown for the front steel arm, and if pressure is applied to the outer curved edges, the jaw of the gable-shaped leaf spring opens. In the rest state, the leaf spring, at its mutually facing hinge areas, exerts a pressure so that the catheter tube excess length can be shut off.

The same applies to the clothes peg 18 whose clamping jaws are held in the closed position in a known manner by means of a leaf spring (not shown) or alternatively by means of the usual clothes peg spring shown here. The two jaws open in a way which is self-evident and familiar to everyone.

FIG. 9 shows a valve of the kind used as a safety valve in many inflatable toys for children. If no anti-parallel pressure is applied from the sides, then the valve remains closed. However, if pressure is applied by the thumb and index finger, for example, the valve lying on the inside, and which does not require further explanation here, is opened, and liquid, in this case urine, can flow out from the opening 101. This kind of opening and closing is familiar even to young children. As an additional safeguard, a stopper 102 is provided which is here held secure by a tab. The tab guides the stopper 102 in a known manner to the correct position for closing the opening 101.

In FIG. 9, the valve is also shown at a far larger scale. Since a considerable air pressure prevails inside a properly inflated inflatable toy animal or in water wings or similar water toys, and a valve such as that in FIG. 9 has to be airtight, it is possible to use simpler designs than those of the known valves according to FIG. 9.

1. Device for voluntary, controlled urethral drainage of urine from the bladder of a human suffering from urinary incontinence, with a tube arrangement which conveys human body fluid and is made of plastic and is physiologically compatible, and with an actuating device for this tube arrangement, characterized by the following features:
a) the tube arrangement is a urinary catheter tube (29) whose length corresponds to the length of the path between the bladder outlet (13) and the urethral outlet (21) and whose external dimension corresponds to the width of the urethra (14), and which catheter tube (29) protrudes beyond this length with an excess length (37),
b) in the area of its excess length (37), the catheter tube (29) has a urine-tight shut-off element (23), (71), (81), (91) (FIGS. 7, 8, 9) which can be opened and closed and has a long period of durability.

2. Device according to claim 1, characterized in that the catheter tube (29) and the catheter tube excess length (37) are made in one piece.

3. Device according to claim 2, characterized in that the catheter tube (29) and the catheter tube excess length (37) have the same external contour.

4. Device according to claim 3, characterized in that they (29, 37) have the same internal contour.

5. Device according to claim 1, characterized in that the catheter tube (29), in its end area remote from the shut-off element, has a position-securing device (33).

6. Device according to claim 2, characterized in that the position-securing device is a circumference-enlarging element (33).

7. Device according to claim 6, characterized in that the circumference-enlarging element is an inflatable balloon element (33).

8. Device according to claim 1, characterized in that the shut-off element is designed as a stopper element (39).

9. Device according to claim 8, characterized in that the stopper element has a grip edge (41).

10. Device according to claim 9, characterized in that the grip edge has a limit stop (47).

11. Device according to claim 9, characterized in that the grip edge (41) also has a sealing action relative to the free end (48) of the excess length (49).

12. Device according to claim 8, characterized in that the stopper element (39) has circumferential ribs (44), (46) on its stopper.

13. Device according to claim 12, characterized in that the circumferential ribs are designed as a thread (92).

14. Device according to claim 1, characterized in that the shut-off element is a spring element having the form of spring clips (71).

15. Device according to claim 14, characterized in that the spring clip is in the nature of an arterial clip.

16. Device according to claim 15, characterized in that the spring clip has the form of a paper clip.

17. Device according to claim 15, characterized in that the spring clip has the form of a small fold-back clip (FIG. 7).

18. Device according to claim 16, characterized in that the spring clip has the form of a small clothes peg (FIG. 8).

19. Device according to claim 1, characterized in that the shut-off element has the form of a nozzle (81) with rotatable plug (82).

20. Device according to claim 1, characterized in that the shut-off element has the form of a closure cuff (23) of the AMS type.

21. Device according to claim 20, characterized in that the “artificial bladder sphincter” of the type AMS 800 (FIG. 1) is not implanted.

22. Device according to claim 1, characterized in that the shut-off element is a screw (91) with screw head, which screw can be screwed an internal thread (94) in the end area of the excess length.

23. Device according to claim 1, characterized in that the shut-off element is a valve (FIG. 9).

24. Device according to claim 23, characterized in that the shut-off element is a valve (FIG. 9) which is of the kind used in a water wing, in inflatable toy animals, or in beach balls, or in similar devices, and opens under a radial actuation force.

25. Device according to claim 25, characterized in that a securing stopper (102) is provided at the end of the valve.

26. Device according to claim 1, characterized in that, in the pinch area (58) of the catheter tube excess length (49), the tube material can be pinched until fluid is closed off, but is elastically resilient.

27. Device according to claim 1, characterized in that a mating piece (38) is provided whose circumference fits into a connector piece (36) of a urinary catheter tube (29) known per se, in that the mating piece having a through-bore merges into a tube area (58) with the pinchable cross section.

28. Device according to claim 28, characterized in that, before the free end area (74), the pinchable cross section has a first limiting rib (63).

29. Device according to claim 29, characterized in that a spring clip (71) (FIGS. 7, 8) is provided upstream of the limiting rib (63).

30. Device according to claim 29, characterized in that a second limiting rib (62) is provided further upstream from the first limiting rib (63), and in that the spring clip (71) (FIGS. 7, 8) sits between the first limiting rib and the second limiting rib.

31. Device according to claim 1, characterized in that the outflow opening (74) of the catheter tube excess length (37) has a detachment edge (76) for the flow of liquid.

32. Device according to claim 1, characterized in that the shut-off element (71) (FIGS. 7, 8) is an element that can be operated by one hand.

33. Device according to claim 33, characterized in that the shut-off element (71) (FIGS. 7, 8, 9) can be operated using one to three fingers.

34. Device according to claim 33, characterized in that the shut-off element has an ergonomic shape which is easy to sterilize.

35. Device according to claim 1, characterized in that the shut-off element can be operated largely free of reaction forces.

36. Device according to claim 1, characterized in that the shut-off element can be operated largely free of torque.

37. Device according to claim 20, characterized in that the closure cuff (23) is joined to a tube (24) which is connected to its interior and which communicates with a fluid reservoir (28).

38. Device according to claim 10, characterized in that the grip edge (41) also has a sealing action relative to the free end (48) of the excess length (49).
